(19) United States
(12) Patent Application Publication
(10) Pub. No.: US 2010/0041769 A1
(43) Pub. Date: Feb. 18, 2010

(54) STABLE AND READY-TO-USE OIL-IN-WATER PROPOFOL MICROEMULSION

(76) Inventors: Ogari Pacheco, Itapira (BR); Roberto Moreira, Itapira (BR); Marisa Rizzi, Itapira (BR)

Correspondence Address: BIRCH STEWART KOLASCH & BIRCH, PO BOX 747, FALLS CHURCH, VA, 22040-0747, US

(21) Appl. No.: 12/447,247

(22) PCT Filed: Oct. 23, 2007
(86) PCT No.: PCT/BR2007/000283

§ 371 Date: Jul. 23, 2009

(30) Foreign Application Priority Data
Oct. 27, 2006 (BR) PI0604377-1

(51) Int. Cl. A61K 31/05; A61P 23/00

(52) U.S. Cl. 514/731

(57) ABSTRACT

The present invention describes a new propofol-containing anesthetic pharmaceutical composition for parenteral administration, in the form of an oil-in-water microemulsion in which the oily phase is constituted by propofol in the form of particles with size comprised between 1 and 100 nm using a single surfactant selected from the group consisting of polyethylene glycol stearates with general formula C₁₇H₃₅COO. (OCH₂CH₂)ₙH or C₁₇H₃₅COO. (OCH₂CH₂)ₙ.COOC₁₇H₃₅.

The anesthetic pharmaceutical composition of the present invention is more potent for induction of hypnosis and anesthesia, has a ready-to-use presentation and highly stable particle size, presenting improved physicochemical properties, and preventing the potential risks of undesirable effects encountered in the state-of-the-art propofol formulations.

### FIELD OF THE INVENTION

The present invention describes an injectable ready-to-use anesthetic pharmaceutical composition for parenteral administration containing propofol as the active agent in the form of an oil-in-water microemulsion, whose disperse hydrophobic particles present much reduced and stable dimensions, which confer a transparent aspect to the microemulsion.

### STATE OF THE ART

The active agent Propofol, the active agent of the present invention, is also designated by the chemical name 2,6-Bis-(1-methylethyl)-phenol. Processes for its preparation are described, for example, in the patents U.S. Pat. No. 2,831,898 and U.S. Pat. No. 4,447,657, whereas its anesthetic and sedative/hypnotic activity in mammals has been first described in the patent U.S. Pat. No. 4,056,635.

An injectable anesthetic lipid emulsion containing propofol at 1% and 2% concentrations (w/v) is currently available in the market under the brand name DRIPIVAN®. In Brazil, this medicine is also found under the brand name PROPOVAN®.

Propofol has a short-term action, being adequate for induction and maintenance of general anesthesia; sedation during local surgical techniques; sedation for ventilated patients receiving intensive care; and conscious sedation for surgeries and diagnostic procedures conducted at intensive care units. It is usually administered by single or repeated intravenous injections by bolus dose or continuous infusion, being rapidly cleared from blood stream and metabolized. For this reason, deep anesthesia is easily controlled and patient recovery after drug withdrawal is usually fast.

Propofol's characteristic of having a rapid start of action is attributed, in great part, to its considerable liposolubility, as this characteristic provides a fast crossing of the hematoencephalic barrier. In order to ensure the adequate velocity for induction of anesthesia, administration of propofol directly to the blood stream is of great interest.

Nevertheless, the low solubility of propofol in water hinders the development of formulations suitable to parenteral administration, as the bodily fluids are basically constituted of water.

A usual technique to increase the hydrophilicity of a certain drug is to obtain ionizable salts derived from this drug in order to turn it more soluble in water, thus allowing the release of the active free base in vivo. However, salt ionization depends on its pKa and, occasionally, the relation between the physiologic pH and the resulting salt pKa may be incompatible with the adequate degree of drug ionization that is necessary for its effective distribution and absorption.

In spite of the significant number of studies using the above-mentioned approach, its application to propofol was not possible. In this case, the propofol-carrier pharmaceutical composition plays a key role on the increase of the hydrosoluble characteristics of the final product in an attempt to provide an adequate drug delivery via blood stream.

The most common technique for development of more adequate formulations for intravenous administration has been the incorporation of propofol to pharmaceutical compositions in the form of oil-in-water emulsions in which the drug is solubilized in the disperse phase, which is usually constituted of fatty acids, vegetable oils and/or triglycerides.

The state of the art presents propofol in oil-in-water emulsions constituted by vegetal oil, preferably soy oil, which incorporate a phospholipid, such as egg lecithin, as a tensoactive agent. As an example, propofol is supplied in an Intralipid® emulsion, a lipid emulsion usually administered intravenously for hypercaloric parenteral nutrition.

Nevertheless, unsaturated fatty acids that constitute the Intralipid® emulsion are highly susceptible to peroxidation, producing hydroperoxides, which may explain the toxicity and carcinogenic risk of such lipid emulsions [Helblock H J; et al “Toxic hydroperoxides in intravenous lipid emulsions used in preterm infants”, Pedaytrics 91, 83 (1993)]. In addition, such emulsions are supportive to microbial growth, leading to the occurrence of postoperative infectious events [Heldmann E et al “The association of propofol usage with postoperative wound infection rate in clean wounds: a retrospective study” Vet Surg 28(4), 1999; Seeberger M D et al “Efficacy of specific aseptic precaution for preventing propofol-related infections: analysis by a quality-assurance programmer using the explicit outcome method”. J Hosp Infect 39(1), 1998; Sklar G E “Propofol and postoperative infections” Ann Pharmacother 31(12). 1997].

Consequently, the oil-in-water emulsions depended on the incorporation of preservatives to prevent the oxidation of the lipid components. The use of preservatives is also necessary to prevent microbial growth or the use of extremely aseptic techniques to avoid the contamination and development of microorganisms in the formulations.

Several documents on the state of the art have shown attempts to prevent or eliminate this problem.

Patents EP 814787 and U.S. Pat. No. 5,714,520 describe a composition containing disodium EDTA in a sufficient amount to prevent microbial growth. Nevertheless, the incorporation of disodium EDTA to a formulation is not recognized by the USP (United States Pharmacopeia) standards as a preventive action against microbial growth [Sklar GE “Propofol and postoperative infections” Ann Pharmacother 31 (12);1521-1523. 1997; and WO 99/39696)].

Patent application WO 99/39696 describes the use of a sulfite, preferably sodium metabisulfite, in a non-toxic amount to delay or suppress the growth of microbial contaminants. However, such substances may cause allergic reactions.

It is important to emphasize that propofol is the drug of choice for use as a long-term sedative medication in bedridden patients. Therefore, the great amount of oil present in these oil-in-water emulsions and the prolonged exposure may cause problems associated to lipid overload in the blood stream, which produces an elevation in lipidemia. This lipid hyperalimentation may exceed the patient capacity to eliminate fats from blood stream resulting in the so-called Fat Overload Syndrome [Lindholm M “Critically ill patients and fat emulsions”, Minerva Anestesiol 58(10), 1992], which causes sudden elevation of triglycerides serum levels, increase of bilirrubin levels in blood stream, “fatty liver”, fever, hepatosplenomegaly, coagulopathy and other dysfunctions in different organs [Haber et al. “Fat overload syndrome. An autopsy study with evaluation of the coagulopathy”. Am J Clin Pathol 90(2):223-227. 1988]. Furthermore, tolerance to fat in some ill patients may decrease, leading to secondary metabolic alterations.

An additional problem related to the use of intravenous emulsions consists in the voluminous dimensions of the particles in the disperse phase (10³ to 10⁴ nm). Very dense particles confer greater instability between the phases and considerable turbidity to formulation. This fact hinders the visual control of sterility of the emulsion and may cause embolism.

The risk posed by the particle size of the emulsions may be overcome by the development of microemulsions. The microemulsions are prepared with the use of surfactants that are adequate for solubilization of the drug under consideration and are able to decrease the interfacial tension.

Patent U.S. Pat. No. 5,637,625 refers to a formulation containing propofol microparticles wrapped by a fat-free and triglyceride-free phospholipid layer. Such microparticles have dimensions between 100 nm and 200 nm. The aqueous phase of the referred formulation is constituted by glucose/phosphate buffer with pH adjusted to 7.0. Although the patent reports that the formulation does not propitiate the development of microorganisms for not containing oils as a microbial nutrient, the inclusion of glucose or another carbohydrate in the formulation accounts to favors microbial growth.

Patent U.S. Pat. No. 6,071,974 incorporates propofol in the solvent 2,5-dimethylisosorbide that is miscible with water. According to the inventors, it constitutes a formulation of comparable efficacy to that of the currently marketed formulation.

Patents U.S. Pat. No. 6,623,765 and GB 2359747 describe injectable microemulsions containing propofol and, additionally, a surfactant constituted by long-chain polymers, specifically poloxamers, for the formation of micelles.

Patent U.S. Pat. No. 6,743,436 describes an injectable composition in the form of oil-in-water microemulsion for propofol incorporating poloxamer as surfactant. The patent refers to poloxamer as an adequate surfactant to form microemulsions with a particle size of 100 nm or below. Additionally, it mentions the use of co-surfactants selected from the group consisting of SOLUTOL HS 15 (Macrogol 15 Hydroxystearate), egg lecithin, LABRASOL (polyoxy caprylic glyceride), polyoxyl 10 oleyl ether, TWEEN, ethanol and polyethylene glycol. According to the patent, poloxamer 407 (0.1 to 5%) is the preferred surfactant, but poloxamer 188 is also mentioned among its examples.

Nevertheless, injectable poloxamer-containing compositions have shown limitations with respect to the use of greater volumes or prolonged administration. These formulations are associated to the Lipid Overload Syndrome, which causes damages to the patient, specifically for causing hypertriglyceridemia and hypercholesterolemia, with consequent induction of atherosclerosis [Jonhston T P et al “Potential downregulation of HMG-CoA reductase after prolonged administration of P-407 in C57BL/6 mice”. J Cardiovasc Pharmacol. 34(6). 1999; Johnston T P et al “Poloxamer 407-induced atherosclerosis in mice appears to be due to lipid derangements and not to its direct effects on endothelial cells and macrophages”. Medaytors Inflamm. 12(3). 2003; Jonhston T P “The P-407-induced murine model of dose-controlled hyperlipidemia and atherosclerosis: a review of finds to date”. J Cardiovasc Pharmacol. 43(4). 2004].

Another propofol microemulsion constituted by a surfactant mixture has been described by Ryoo et al. [Ryoo H K et al “Development of propofol-loaded microemulsion system for parenteral delivery”. Arch Pharm Res. 2005 28(12):1400-1404]. In their study, Ryoo et al. presented an accelerated stability assay (at 40° C.) conducted within only 8 weeks involving three different 1% propofol microemulsions containing a surfactant/co-surfactant mixture comprised by Solutol/Ethanol in a 5:1 ratio. The studied microemulsions were prepared containing 4, 6 and 8% by weight of the 5:1 Solutol/Ethanol mixture. The graph presented in that study shows that two out of three formulations demonstrated an increase in particle diameter to a value close to 100 nm in one case (mixture at 6%) and close to 200 nm in the other case (mixture at 4%) within only 8 weeks of follow-up of the stability assay. In addition of containing a surfactant mixture, this formulation is not able to maintain the desired stability regarding particle size.

Patent application WO2005/079758 describes bases for a self-microemulsifiable composition comprising two or four components. The base that comprehends two components is constituted by a first component formed by a surfactant containing polyethylene glycol and a liquid propofol containing vitamin E; and by a second component that is a saline isotonic aqueous carrier, or dextrose. The carrier is mixed to the first component at the moment of use. On the other hand, the base comprising four components is more complex, being constituted essentially by a surfactant containing polyethylene glycol, a vitamin E-containing liquid propofol, a co-solvent immiscible in water, and ethanol. The document affirms that the base is stable for undetermined time; however, no stability assay is presented to confirm this statement. One of the advantages attributed to this invention would be the possibility of preparing a formulation containing propofol at high concentrations. Nevertheless, the author does not justify the importance of increasing propofol concentration. On the contrary, it is known that high concentrations of propofol may cause side effects characterized, for example, by myocardial failure, metabolic acidosis and rabdomyolisis [De Cosmo, G et al. “Sedation in PACU: The Role of Propofol” Current Drug Targets. 2005. 6 (7): 741]

In view of the deficiencies found in the state of the art referring to the excipients employed in injectable propofol compositions, there is the need for development of ready-to-use microemulsions that present smaller particle size and high stability and that minimize the side effects inherent to propofol or those related to Overload Syndromes.

### DESCRIPTION OF THE INVENTION

In this context, the present invention describes an injectable anesthetic pharmaceutical composition, containing propofol as the active agent, in the form of a highly stable ready-to-use oil-in-water microemulsion, whose disperse hydrophobic particles present much reduced dimensions.

Surprisingly, the propofol microemulsion of the present invention was more potent for induction and maintenance of hypnosis and anesthesia compared to the propofol compositions existing in the prior art. It was noticed that the ready-to-use microemulsion of the present invention produces anesthetic and hypnotic effects equivalent to those obtained with the conventional formulations containing 1% (w/v) propofol, though with half the concentration (0.5% w/v). The use of lower propofol doses to reach the desired anesthetic and hypnotic effect minimizes the risks of side effects and potential adverse reactions inherent to propofol.

Therefore, the microemulsion of the present invention is characterized by the propofol being comprised in the range between 0.1 and 5% (w/v) of the final composition. Preferably, propofol is comprised in the 0.1 to 2% (w/v) range and, even more preferably, propofol is comprised in the range between 0.5% and 1% (w/v) of the final composition.

An important aspect of the ready-to-use microemulsion of the present invention, resides in the fact that its disperse particles present much reduced dimensions, comprehended between 1 and 100 nm, more specifically, between 1 and 50 nm, which remained stable for at least 12 months. Due to this characteristic, the microemulsion of the present invention has a transparent aspect and viscosity comparable to that of an aqueous solution.

The microemulsion of the present invention is also characterized by containing a single surfactant selected from the group consisting of polyethylene glycol stearates, preferably non-ionic. Its general formula is C₁₇H₃₅COO.(OCH₂CH₂)ₙH or C₁₇H₃₅COO.(OCH₂CH₂)ₙ.COOC₁₇H₃₅ and it is comprised within the range between 1 and 50% (w/v) of final composition, or preferably between 5 and 20% (w/v) of the final composition.

Preferably, the microemulsion of the present invention uses SOLUTOL HS 15 (Macrogol 15 Hydroxystearate) as surfactant, but it is not restrictive to the scope of the present invention. Other surfactants adequate for the development of the present invention are those comprised in the group consisting of polyoxyethylene (4) monostearate, polyoxyethylene (6) monostearate, polyoxyethylene (8) monostearate, polyoxyethylene (12) monostearate, polyoxyethylene (20) monostearate, polyoxyethylene (30) stearate, polyoxyethylene (40) monostearate, polyoxyethylene (50) monostearate, polyoxyethylene (100) monostearate, polyoxyethylene (150) stearate, polyoxyethylene (4) distearate, polyoxyethylene (8) distearate, polyoxyethylene (12) distearate, polyoxyethylene (32) distearate, polyoxyethylene (150) distearate.

Optionally, the microemulsion of the present invention may contain agents for adjustment of pH and agents for adjustment of osmolarity that are pharmaceutically acceptable for the intravenous environment. The preferred agents for such purposes, but not limiting the scope, are sodium hydroxide and glycerol, respectively.

A microemulsion of the present invention may be sterilized by filtration through a sterilizing membrane with 0.22 μm-diameter pores.

The administration route of the pharmaceutical composition of the present invention is, preferably, the intravenous route. Nevertheless, the formulation is also adequate for intramuscular, subcutaneous, intradermal and spinal administration.

One aspect of the present invention is that the pharmaceutical composition obtained by the ingredients and processes described herein provides a thermodynamically stable product with a single homogeneous phase and transparent appearance.

Another aspect of the present invention is that the microemulsion described herein is highly stable and ready-to-use, with particle size maintained below 50 nm, even after the 12-month duration of the stability assay, unlike the most similar microemulsions known in the state of the art.

The microemulsion of the present invention may be prepared by conventional techniques for preparing microemulsions described in the state of the art. Preferably, the microemulsion of the present invention may be prepared according to the following steps:

(a) Providing a first receptacle containing an amount between 1 to 50% (w/v) of a non-ionic polyethylene glycol stearate surfactant, maintaining the system under constant stirring, preferably under heating around 50° C., until fusion of the surfactant;

(b) Adding approximately 5-10% of the total water of the final composition and an amount between 0.1 and 5% (w/v) of propofol to the mixture of the first receptacle, maintaining the system under constant stirring;

(c) Providing a second receptacle with a stirring system containing 50-85% of total water for the final composition;

(d) Adding the mixture of the first receptacle to the second receptacle, under constant agitation, until homogenization;

(e) Completing the final volume of the composition with water, under constant agitation, until homogenization;

(f) Sterilizing the final composition using a 0.22 μm filtrating membrane.

Optionally, a pharmaceutically acceptable osmolarity agent can be added to the mixture together with the accomplishment of the step (d).

Yet, a pharmaceutically acceptable pH adjustment agent can be added during the step (e) to provide a final pH between 5.0 and 8.5.

In addition to the physicochemical improvements and the surprising verification of the increase of the power for the anesthetic and hypnotic effect obtained with the above-mentioned pharmaceutical composition of the present invention, the microemulsion of the present invention has also other advantages over the compositions known in the state of the art, such as: (i) the fact that the composition of the present invention does not present lipid- or lecithin-derived components eliminates the risks of formation of toxic sub-products from degradation/oxidation of these components; (ii) the composition of the present invention does not present components derived from oils and sugars, which are factors that favor the growth of microorganisms and, as result, the potential contamination of the product and patients is minimal; (iii) when administered during prolonged periods, the composition of the present invention eliminates the damage associated with the lipid Overload Syndrome; (iv) because the composition of the present invention has surfactant of another nature, it also eliminates the damages associated with the Lipid Overload Syndrome that can occur with poloxamer-containing formulations administered for a prolonged period; (v) because the composition of the present invention presents particles of much reduced dimensions, it eliminates the risks associated with embolism; (vi) because it requires half the dose to reach the desired anesthetic and hypnotic effects with propofol, the composition of the present invention minimizes potential side effects and adverse reactions associated with higher doses of propofol used until the present moment; (vii) the highly stable and ready-to-use microemulsion of the present invention can be stored for long periods without undergoing alterations in its physicochemical aspect and presenting easiness of handling and administration to patient.

Therefore, the pharmaceutical composition of the present invention permits either short-term or long-term parenteral use of propofol, with increased safety and reliability.

The following Examples aim to demonstrate, in a non-restrictive manner, the best form for performing the present invention, as well as its advantages in comparison to the state-of-the-art formulations.

### Example 1

### Preparation of 1% and 0.5% Propofol-Containing Microemulsions

One accomplishment of the present invention may be illustrated by preparation of MICROEMULSION 1 and MICROEMULSION 2, whose preferred propofol and excipient ratios are described in Table 1 below:

TABLE 1
Propofol microemulsions.

| | MICROEMULSION 1 | MICROEMULSION 2 |
|---|---|---|
| Components | % w/v | % w/v |
| Propofol | 1 | 0.5 |
| Macrogol 15 Hydroxy- | 10 | 10 |
| stearate (SOLUTOL | | |
| HS 15) | | |
| Glycerol (Glycerin BCA | 2.5 | 2.5 |
| FCTA) | | |
| Sodium Hydroxide PA | q.s.t. pH 5.0 to 8.5 | q.s.t. pH 5.0 to 8.5 |
| Water for Injection | q.s.t 100 | q.s.t. 100 |

According to the present invention, MICROEMULSIONS 1 and 2 were prepared as follows:

Seventy percent of the total water for injection was added to a stainless steel reactor presenting a stirring system. Separately, the macrogol 15 hydroxystearate (SOLUTOL HS 15) was added to a stainless steel receptacle and heated up to 50° C., under constant stirring, for complete fusion of the product. Next, 6% of the total water for injection and propofol were added to the fused surfactant, under constant agitation.

The content of the stainless steel receptacle was added to the reactor containing 70% of the total water for injection, together with glycerol, under constant stirring, until total homogenization was obtained. The pH was adjusted to the 5.0 to 8.5 range, using a 1 N sodium hydroxide solution, previously prepared with water for injection. The final volume of the composition was completed with water for injection, checking again the final pH.

The final composition was filtered in AP-15 257-25 Pre-Filter and GVWP 293-25 sterilizing membrane with 0.22 μm-diameter pores.

At the end of this process, the product was enclosed in appropriate sterile flasks.

The resulting formulation presents as a transparent microemulsion free of foreign particles.

### Example 2

### Stability of the Propofol Microemulsion of the Present Invention

Freshly prepared MICROEMULSIONS 1 and 2 according to Example 1 were submitted to tests to evaluate their characteristics. The evaluation of particle size was the most important issue for the present invention because the stabilization of this parameter is one of the deficiencies of the propofol microemulsions described in the art.

Particle size was monitored during the 12-month stability assay under normal temperature conditions (Table 2) and during the 180-day accelerate stability assay at 40° C. (Table 3).

The results displayed on Tables 2 and 3 demonstrate that there was no significant alteration in particle size during the monitoring time. Even the older samples presented significantly smaller particle size than the maximum limit of 50 nm established by the present invention as the preferential limit.

TABLE 2
Effect of the storage time, under the usual conditions of long-term stability
assays, on the particle size of the propofol microemulsions.

| | Particle sizes (established limit: up to 100 nm) | | | | | |
|---|---|---|---|---|---|---|
| | 30° C. ± 2° C. and relative humidity of 75% ± 5% | | | | | |
| | | 1 | 3 | 6 | 9 | 12 |
| Microemulsion | INITIAL | MONTH | MONTHS | MONTHS | MONTHS | MONTHS |
| 1 | 16.2 | 17.5 | 17.1 | 19.2 | 17.3 | 16.6 |
| 2 | 19.2 | 29.6 | 36.0 | 27.1 | 17 | 18.8 |

TABLE 3
Effect of the storage time, under the usual conditions of accelerate
stability assays, on the particle size of the propofol microemulsions

| | Particle sizes (established limit: up to 100 nm) | | | | | |
|---|---|---|---|---|---|---|
| | 40° C. ± 2° C. and relative humidity of 75% ± 5% | | | | | |
| | | 1 | 3 | 6 | 9 | 12 |
| Microemulsion | INITIAL | MONTH | MONTHS | MONTHS | MONTHS | MONTHS |
| 1 | 19.2 | 29.6 | 36.0 | 27.1 | 17 | 18.8 |
| 2 | 19.2 | 29.6 | 32.1 | 37.6 | 28.9 | 19.2 |

### Example 3

### Comparative Analysis of the Stability of State-of-the-Art Propofol Microemulsions and the Microemulsion of the Present Invention

Formulations described in the reference Patent U.S. Pat. No. 6,743,436 (Examples 1, 5 and 6) were elected for comparative purposes as well as to demonstrate the physicochemical improvements reached with the pharmaceutical composition of the present invention, considering the teachings of the closest state-of-the-art to this new composition.

When the compositions were reproduced according to the teachings of the Patent used as a reference it was observed that, in general, the turbidity did not meet the microemulsion parameters and the analysis demonstrated particle size much above the limit of 100 nm (as shown in Table 4).

TABLE 4
Determination of the particle sizes obtained in accelerated
stability assays (40° C. ± 2° C.).

| | | | |
|---|---|---|---|
| INITIAL | 30 DAYS | 60 DAYS | 90 DAYS |

Specification: Particle Size (up to 100 nm)

| | | | | |
|---|---|---|---|---|
| Example 1 * | 571.4 | 814.2 | 877.1 | 669.6 |
| Example 5 * | 1755.8 | 1543.6 | 2218.8 | 1873.4 |
| Example 6 ** | 33.2 | 30.5 | 36.6 | 38.4 |

* Poloxamer as surfactant in combination with Solutol HS 15 as co-surfactant;
** Poloxamer in combination with co-surfactants other than Solutol HS 15.

In contrast with these results, the composition of the present invention, as observed in the Example 2 of the present invention, was completely transparent and its particle size, even after 12 months of stability monitoring, remained stable, with dimensions significantly below 50 nm. This clearly demonstrates the physicochemical improvements reached with the pharmaceutical composition of the present invention.

### Example 4

### Assessment of Sterility of the Present Invention Microemulsion (Microemulsion 1)

The microemulsion prepared according to the Example 1 of the present invention was submitted to a sterility assay by incubation in three different culture media, under specific temperature conditions that are adequate for microbiological growth in each culture medium, observing whether or not development of colony forming units (c.f.u.) and turbidity of the medium occurred within 14 days of incubation. The culture media used are presented in the following table:

TABLE 5
Culture media used for the sterility assay with
MICROEMULSION 1 of the present invention.

| | INCUBATION | INCUBATION |
|---|---|---|
| CULTURE MEDIUM | TEMPERATURE | TIME |
| Tryptic Soy Broth (TSB) | 25° C. | 14 days |
| Fluid Thyoglicollate | 35° C. | 14 days |
| Medium (THIO) | | |
| Peptone Water | 25° C. | 14 days |

After the incubation period, no signs of bacterial growth were observed in any of the culture media, confirming the safety of the formulation under the perspective of sterility.

### Example 5

### Comparison of Pharmacological Parameters Observed with 1% Microemulsion, 0.5% Microemulsion and 1% Commercial Emulsion

In vivo assays were performed to compare the pharmacological parameters of the present invention formulations, at 1% and 0.5% concentrations, to the pharmacological parameters presented by the 1% commercially available emulsion (Propovan®). The hypnotic and anesthetic activities were investigated by means of intravenous infusion in 6 rats weighing 260 to 350 g. The animals were anesthetized by ethylic ether inhalation and, subsequently, placed in ventral decubitus. An incision was made at the anterior cervical region and the jugular vein was dissected. A catheter filled with heparin was tunneled through the subcutaneous cell tissue up to the posterior cervical region where it was fixed to the skin. The assay was performed only when the animal presented complete recovery from ether anesthesia (±60 min). The propofol formulations were infused during 1 hour through an infusion pump (B. Braun) at a volume of 40 μL/min. The hypnotic and anesthetic latencies were determined by the analysis of the time elapsed between the beginning of drug infusion and the loss of postural reflex and absence of reaction to painful stimulus (pressure of the skin on the planar region of the hindfoot), respectively. The obtained results are displayed in Table 6, below:

TABLE 6
Comparison of the pharmacological parameters observed
with a commercial 1% propofol emulsion (w/v) (Propovan ®),
MICROEMULSION 1 and MICROEMULSION 2.

| | Result (n = 6; p < 0.05) | | |
|---|---|---|---|
| | Propo- | MICRO- | MICRO- |
| PARAMETER | van ® 1% | EMULSION 1 | EMULSION 2 |
| Hypnotic Latency (min) | 2.0 ± 0.2 | 1.6 ± 0.1 | 3.5 ± 0.4 |
| Hypnotic Dose (mg/kg) | 5.8 ± 0.8 | 4.8 ± 0.4 | 5.1 ± 0.7 |
| Anesthetic Latency (min) | 6.5 ± 0.6 | 3.4 ± 0.3 | 8.2 ± 0.7 |
| Anesthetic Dose (mg/kg) | 18.6 ± 1.9 | 10.4 ± 0.8 | 11.5 ± 0.9 |
| Anesthetic | 5.8 ± 0.3 | 26.6 ± 9.1 | 5.0 ± 0.6 |
| Recovery (min) | | | |
| Hypnotic Recovery (min) | 24.8 ± 1.8 | 42.9 ± 9.6 | 20.6 ± 1.1 |

The comparative results presented in Table 6 above showed that MICROEMULSION 1 of the present invention presented lower hypnotic and anesthetic latency, lower dose to reach hypnotic and anesthetic effect and longer recovery time from hypnosis and anesthesia, compared to parameters found for the commercially available propofol composition de (Propovan®) with the same propofol concentration (1% w/v). Table 6 also demonstrates that pharmacological parameters equivalent to those obtained with the 1% (w/v) propofol-containing emulsion Propovan® may be reached with MICROEMULSION 2 of the present invention, which presents half the propofol concentration (0.5% w/v) of the commercially available emulsion.

These findings clearly demonstrate that the propofol microemulsions prepared according to the present invention resulted, in an unexpected and not yet reported manner, in greater hypnotic and anesthetic power when compared to commercially available or state-of-the-art propofol formulations.

1.-26. (canceled)

27. An transparent, stable, ready-to-use oil-in-water propofol microemulsion comprising propofol in an amount ranging from 0.1% to 5% w/v, a surfactant selected from the group consisting of polyethylene glycol stearates with the general formula C17H35COO.(OCH2CH2)nH or C17H35COOO. (OCH2CH2)n.COOC17H35 in an amount ranging from 1% to 50% w/v, and water for injection to complete 100% w/v of the final composition, having particle size ranging from 1 to 100 nm and a pH in the range from 5.0 to 8.5.

28. The oil-in-water propofol microemulsion according to claim 27 in which the propofol is present in an amount ranging from 0.1% to 2% w/v.

29. The oil-in-water proporfol microemulsion according to claim 27 in which the propofol is present in an amount ranging from 0.5% to 1% w/v.

30. The oil-in-water propofol microemulsion according to claim 27 in which the particle size ranges from 1 to 50 nm.

31. The oil-in-water propofol microemulsion according to claim 27 in which the surfactant is macrogol 15 hydroxystearate.

32. The oil-in-water propofol microemulsion according to claim 31 in which the macrogol 15 hydroxystearate is present in an amount ranging from 5% to 20% w/v.

33. The oil-in-water propofol microemulsion of claim 27 comprising:
propofol in an amount ranging from 0.5% to 1% w/v;
macrogol 15 hydroxystearate as a surfactant in an amount of 10% w/v;
glycerol as an osmolarity agent in an amount of 2.5% w/v;
sodium hydroxide in an amount sufficient to provide a pH in the range from 5.0 to 8.5; and
water for injection to complete 100% w/v of the final composition,
having particle size ranging from 1 to 50 nm.

34. A process for preparing a transparent oil-in-water propofol microemulsion comprising the following steps:
(a) Providing a first receptacle containing an amount from 1% to 50% w/v, based on final volume of the composition, of a non-ionic polyethylene glycol stearate surfactant, maintaining the system under constant stirring, preferably under heating at 50° C., until fusion of the surfactant;
(b) Adding water for injection in the amount of 5-10% of the volume of the final composition and an amount from 0.1 to 5% w/v, based on the final volume of the composition, of propofol to the content of the first receptacle, maintaining the system under constant stirring;
(c) Providing a second receptacle with a stirring system containing 50-85% of the total water for injection for the final composition;
(d) Adding the mixture of the first receptacle, together with glycerol, to the second receptacle, under constant stirring, until homogenization;
(e) Completing the final volume of the composition with water for injection, under constant stirring, until homogenization;
(f) Sterilizing the final composition by filtering.

35. The process according to claim 34 further comprising adding a pharmaceutically acceptable pH adjustment agent during the step (e), to provide a final pH ranging from 5.0 to 8.5.

36. The process according to claim 35 in which the pH adjustment agent is sodium hydroxide.

37. An oil-in-water propofol microemulsion prepared by the process according to claim 36.

38. A method of inducing and/or maintaining hypnosis, general anesthesia and/or sedation in a mammal, comprising administering to said mammal an effective amount of an oil-in-water propofol microemulsion of claim 27.

39. A method of inducing and/or maintaining hypnosis, general anesthesia and/or sedation in a mammal, comprising administering to said mammal an effective amount of an oil-in-water propofol microemulsion of claim 33.
